(19) United States
(12) Patent Application Publication
(10) Pub. No.: US 2022/0211698 A1
(43) Pub. Date: Jul. 7, 2022

(54) ANTI MALARIAL COMPOUNDS

(71) Applicant: INSTITUTO DE MEDICINA MOLECULAR, Lisboa (PT)

(72) Inventors: Sonali SETUA, Lisboa (PT); Maria Manuel DIAS DA MOTA, Lisboa (PT); Francisco Javier ENGUITA, Lisboa (PT); Varadharajan SUNDARAMURTHY, Bangalore (IN); Harish RANGA-PRASAD, Victoria (CA)

(73) Assignee: INSTITUTO DE MEDICINA MOLECULAR, Lisboa (PT)

(21) Appl. No.: 17/577,643

(22) Filed: Jan. 18, 2022

Related U.S. Application Data
(63) Continuation-in-part of application No. PCT/US21/59700.
(60) Provisional application No. 63/114,743, filed on Nov. 17, 2020.

(51) Int. Cl. A61K 31/496; A61P 33/06

(52) U.S. Cl. CPC A61K 31/496; A61P 33/06

(57) ABSTRACT

Disclosed herein are compositions and methods to prevent Plasmodium parasites from evading host cell autophagy responses, including by blocking binding of Plasmodium Upregulated in infective sporozoites 3 (UIS3) to host cell autophagy proteins, such as Microtubule-associated protein 1A/1B-light chain 3 (LC3).

### CROSS REFERENCE TO RELATED APPLICATIONS

This application is a continuation in part of International Application No. PCT/US2021/059700, which was filed on Nov. 17, 2021, and claims priority to U.S. provisional Application No. 63/114,743, which was filed Nov. 17, 2020. These applications and incorporated herein by reference.

### SEQUENCE LISTING

The Sequence Listing submitted herewith, (2022-01-18_CIP_Sequence_listing_ST25), via EFS-Web, is an ASCII text file created on Jan. 18, 2022, and is 4,669 bytes is hereby incorporated by reference.

### FIELD OF THE INVENTION

The inventions described here relate to compositions and methods to prevent Plasmodium parasites from evading host cell autophagy responses.

### BACKGROUND

For intracellular pathogens, the host cell acts as the replicative compartment that provides the pathogen with the necessary resources such as nutrients and, energy, required for their development as well as shields them from the components of the host immune system such as complements. However, host cells are equipped with various intracellular defense mechanism(s) that can destroy the invading pathogen. Autophagy is emerging as one of the most remarkable tools of the intracellular defence weaponry that pathogens must confront upon host cell invasion.

Autophagy is a conserved cell-autonomous catabolic process responsible for the degradation and recycling of intracellular components to maintain cellular homeostasis. Upon initiation of canonical autophagy, a double membrane autophagosome forms around the cellular materials that need to be degraded. The sequential activation of ubiquitin-like protein-machinery, such as Atg7 (E1-like), Atg3 (E2-like), and Atg12-Atg5-Atg16L1 (E3-like) complex associates LC3 (Microtubule-associated protein 1A/1B-light chain 3, a member of Atg8 protein family) with the autophagosome isolation membrane. Membrane-bound LC3 binds autophagy adaptor proteins on the cargo which leads to engulfment of the cargo by the autophagosomal membrane followed by its lysosomal degradation. (Yu et al.) Although initially demonstrated as a response to cellular stress, autophagy is now a well-established host defence mechanism that can significantly hinder the virulence of the intracellular pathogens. (Levine et al.)

However, intracellular organisms have evolved various sophisticated strategies to manipulate this host cell pathway to their advantage or escape their recognition and capture by the autophagy machinery, thus preventing pathogen elimination from the host cell. (Wu et al., Choi et al., Latre de Late). For example, the intracellular bacteria Shigella residing in its vacuolar compartment only for a short duration secretes an effector protein that surrounds the bacteria while in the host cell cytosol and impairs their detection by the host autophagy machinery. (Ogawa et al.) However, other bacteria such as Coxiella burnetii replicate in an autophagosome-like vacuolar compartment by delaying lysosomal fusion with it. (Ramano et al.) Several viruses, such as herpes simplex, block the initiation of autophagy flux by modulating host signalling pathway. (Orvedahl et al.) In the case of the apicomplexan Toxoplasma gondii, the host autophagy response is inhibited by two microneme proteins, MIC3 and MICE, which activate the host EGFR/Akt signalling pathway and thereby protect its vacuolar compartment from fusing with the lysosome. (Muniz-Feliciano et al.)

Apicomplexan parasites such as Plasmodium spp., Toxoplasma, and Theileria are a class of intracellular pathogens that are responsible for several important human diseases. As obligatory intracellular organisms, they maintain a complex interplay of host-pathogen interaction with autophagy playing a critical role in this process. (Latre de Late, Evans et al.) Among these, Plasmodium spp., the causative agent of malaria, demands special attention due to the enormous global health and economic burden caused by this disease. (Ashley et al.) While precise figures for malaria's impact are not known, the World Health Organization (“WHO”) estimates ^{˜}250 million cases and ¹⁸ 500 thousand deaths per year due to malaria. (WHO World malaria report 2018)

Five Plasmodium species—P. falciparum, P. vivax, P. ovale, P. malariae and P. knowlesi—are known to cause human malaria. The first two species are responsible for most infections worldwide. P. falciparum infections result in more than 90% of the deaths. P. vivax and P. ovale have dormant liver stage parasites forms, called “hypnozoites”, which can reactivate, or relapse, and cause malaria several months or years after the initial infection. Plasmodium poses a complex life cycle that involves a mosquito vector and the mammalian host as well as infection of various cell types within the same mammalian host. Liver-stage infection is the first and obligatory step of malaria infection in mammals where Plasmodium sporozoite matures into thousands of pathogenic merozoites within the confinement of the parasitophorous vacuole membrane (PVM) inside hepatocytes. (Vaughn & Kappe) Although traditionally termed as the silent stage of malaria infection, recent pieces of evidence proved that the host cell could sense Plasmodium parasites and trigger a response against them. (Liehl et al., Real et al.) Indeed, liver-stage Plasmodium PVM gets decorated with LC3-containing autophagy-derived vesicles as well as LC3 binding proteins such as p62, NBR1, and NDP52 along with ubiquitin. (Thieleke-Matos et al., Schmuckli-Maurer et al.) Moreover, vesicles positive for LAMP1, a lysosomal marker protein, proposed to be used as a nutrient source by the parasite for its fast development inside hepatocytes, surround Plasmodium throughout infection without fusing with the PVM and triggering a host autaphagy response (Lopes da Silva et al.). Thus, parasites can evade the host autophagy response. Furthermore, while it has been proposed that parasite redirect PVM-associated LC3 towards the tubulovesicular network (TVN) in later stages of intrahepatic development to avoid elimination by the host cells (Agop-Nersesian et al.), Plasmodium PVM-resident protein UIS3 (Upregulated in infective sporozoites 3) interacts with LC3 from very early stages of infection and thereby prevents parasite elimination from the cells by the host autophagy machinery (Real et al.).

UIS3 is one of the very few liver-stage PVM proteins, identified so far, whose molecular function is known. UIS3 is essential for Plasmodium liver stage development, as a mutation of this protein results in parasite elimination at early stages of infection (Mueller et al.). In their study, Real et al. have presented several pieces of evidence that confirmed the protective function of UIS3 during Plasmodium's intrahepatic development (Real et al.). While UIS3-deficient parasites cannot develop in wild type hepatocytes, they develop inside autophagy-deficient host cells which strongly suggests that UIS3 plays a critical role in the interaction between the parasite and the host autophagy pathway. Using exogenously expressed UIS3 in HeLa cells, UIS3 directly binds to LC3, possibly through its non-canonical LIR motif. Furthermore, by molecular docking analysis and in vitro experiments, the residues on UIS3 potentially involved in this interaction have been identified. Together, these data indicate that by sequestering LC3 on the PVM, UIS3 blocks its binding to other autophagy target proteins such as P62 and Rab7 effector proteins leading to the inhibition of host autophagy defense response. (Farre & Subramani, Ganley et al.) Here, this disclosure describes small molecule inhibitors of the UIS3's interaction with LC3, which prevent Plasmodium parasites from evading the host autophagy response. These inhibitors are useful in effective anti-malarial strategies, which impede Plasmodium infection without interfering with intrinsic host cell functions.

### SUMMARY OF THE INVENTION

The invention described here relates to methods for treating Plasmodium infections with small molecule compounds that disrupt direct interactions between host cell LC3 and parasite UIS3 proteins. Accordingly, the invention includes compositions of, and methods for administering small molecule drugs that treat Plasmodium infections by inhibiting sequestration of host cell LC3 on the surface of PVMs by Plasmodium UIS3, thereby allowing the host cell to eliminate the parasite by an autophagy-dependent process.

In some compositions and methods of the invention, the small molecule drug that inhibits LC3 sequestration by UIS3 is a phenyloxadiazole compound. More particularly, in certain compositions and methods of the invention, the phenyloxadiazole compound has an oxadiazole ring connected to a tri-fluoro-methyl-benzene and an N-alkyl-piperazine, linked to a nitrile derivative of benzoic acid. For example, the phenyloxadiazole compound inhibitor of the invention may be (4-{[4-(4-{5-[3-(trifluoromethyl) phenyl]-1,2,4-oxadiazol-3-yl}benzyl)piperazino]carbonyl}benzonitrile (“C4”).

### BRIEF DESCRIPTION OF THE FIGURES

FIGS. 1A-3 depict steps of an exemplified virtual library screen (VLS) for small molecule compounds that bind P. falciparum UIS3 at its LC3 binding site, and a phenotypic screen (PHS) of the selected compounds for activity against Plasmodium at the liver stages of Plasmodium infection.

FIG. 1A is a schematic representation of the different steps involved in VLS using ZINC database for P. falciparum with the number of the compounds identified in each phase.

FIG. 13 is a schematic representation of the experimental steps involved in PHS of selected compounds against P. berghei (bi) or PfUIS3@Pb (bii) infection in Huh7 cells.

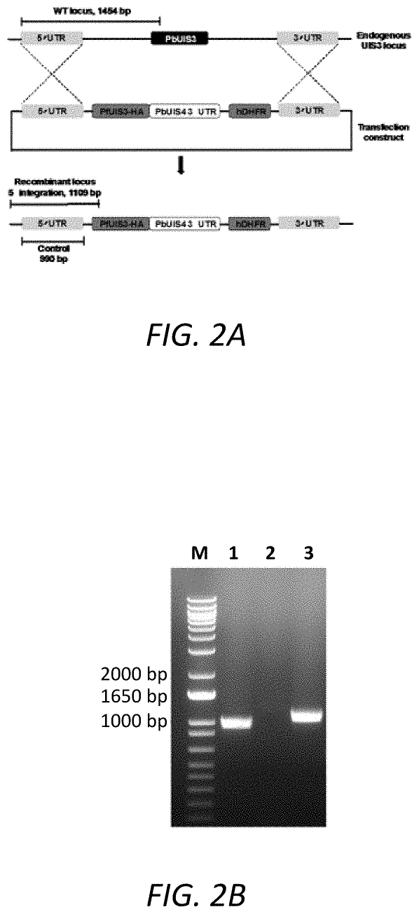

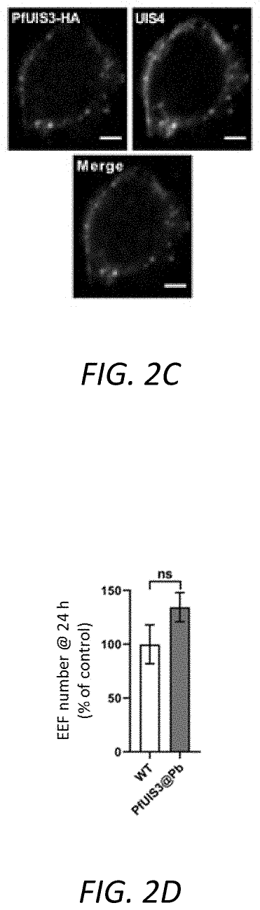

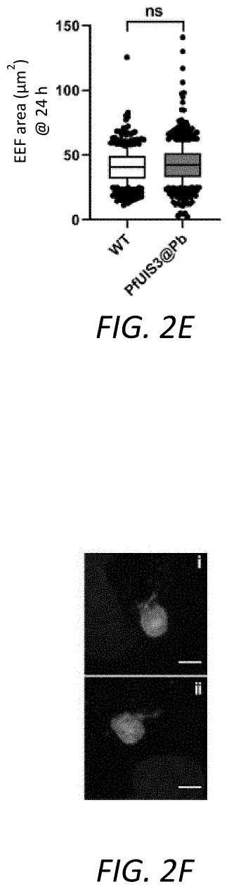

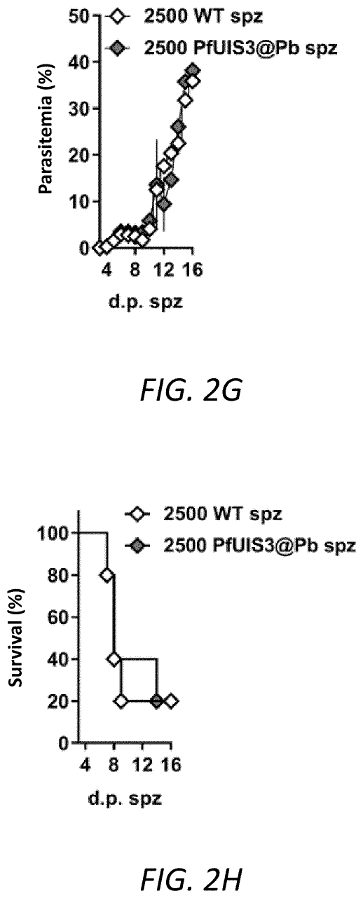

FIGS. 2A-H relate to the generation and characterization of PfUIS3@Pb.

FIG. 2A is a schematic representation of the approach used for replacing UIS3 gene of P. berghei ANKA (507cl1) with that of P. falciparum 3D7, marked with 2 copies of HA tag. The location of the different primers pairs used to verify recombination by PCR and the expected length of the amplicons are indicated.

FIG. 2B shows PCR analysis confirmation of the integration of the PfUIS3-HA replacement fragment in genomic DNA from PfUIS3@Pb parasites. 1: Control (5′ UTR of PbUIS3 gene); 2: WT locus; 3: PfUIS3-HA recombinant locus 5′ integration. PCR primers are listed in Tables 2 and 3. Control (5′ UTR of PbUIS3 gene).

FIG. 2C shows PVM localization of PfUIS3-HA in representative confocal images, based on two independent experiments, of HuH7 cells infected with PfUIS3@Pb parasites. 24 post-infection cells were fixed and immunostained with anti-HA (green), anti-UIS4 (red) and Hoechst (blue). Scale bars=2 μm.

FIG. 2D compares the EEF numbers of WT and PfUIS3@Pb parasites at 24 h post-infection. Data represent means±SEM (n=2). Statistical significance was assessed using the non-parametric two-tailed Mann-Whitney test. ns: non-significant.

FIG. 2E compares EEF sizes of WT and PfUIS3@Pb parasites at 24 h post-infection. Data represent means±SEM (n=2). Statistical significance was assessed using the non-parametric two-tailed Mann-Whitney test. ns: non-significant.

FIG. 2F shows a comparison of EEF sizes in representative confocal images of HuH7 cells infected with (i) WT and (ii) PfUIS3@Pb. Scale bars=5 μm.

FIG. 2G compares blood parasitaemia of mice infected with 2500 sporozoites from WT or PfUIS3@Pb parasites. Data represent mean (of 5 mice in each group)±SEM. d.p.spz: day post-sporozoite.

FIG. 2H compares survival of mice infected with 2500 sporozoites from WT or PfUIS3@Pb parasites. Data represent mean (of 5 mice in each group)±SEM. d.p.spz: day post-sporozoite.

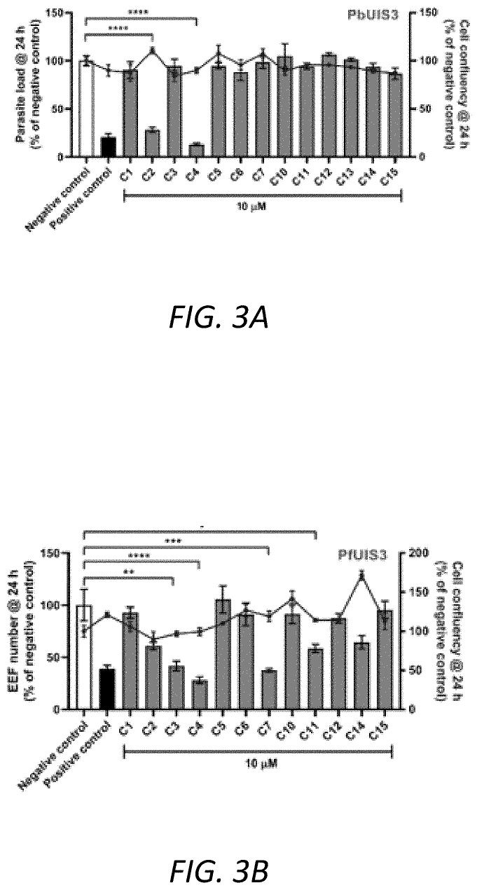

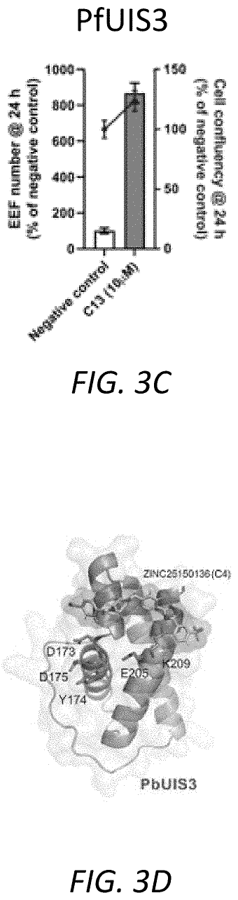

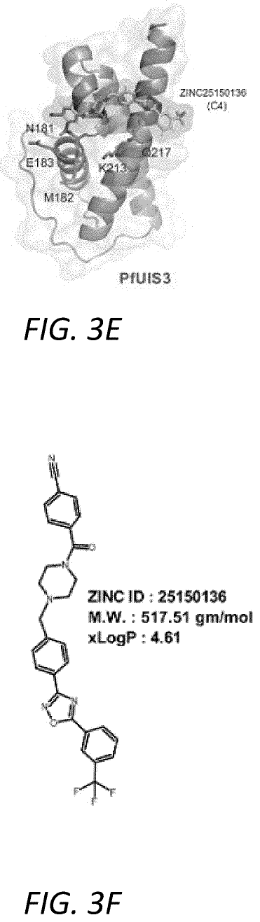

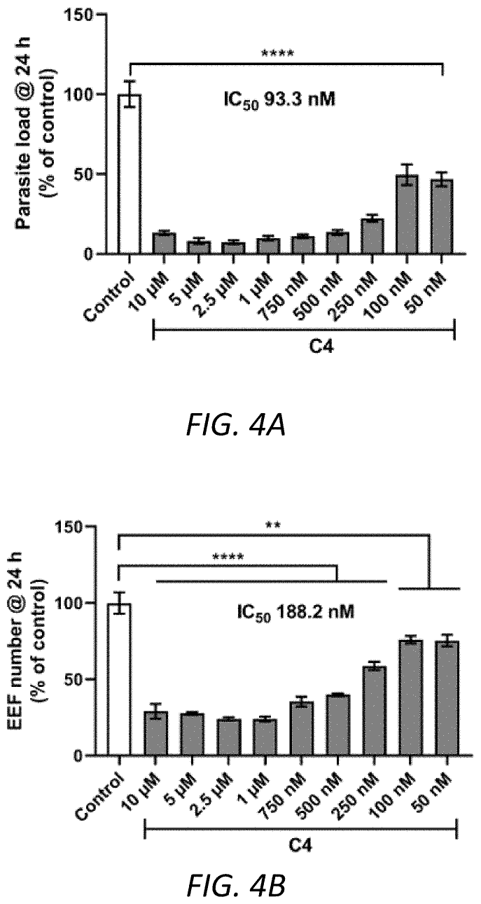

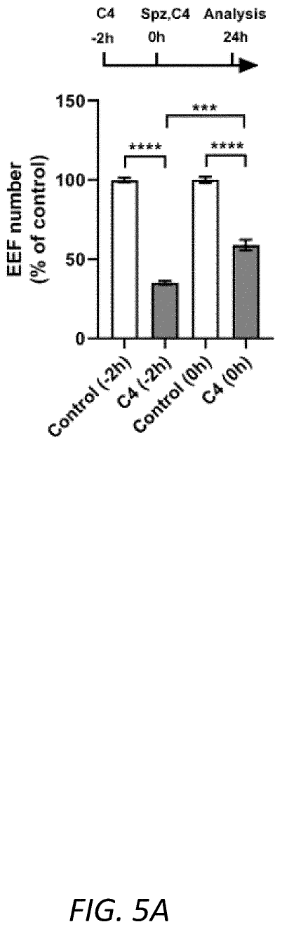

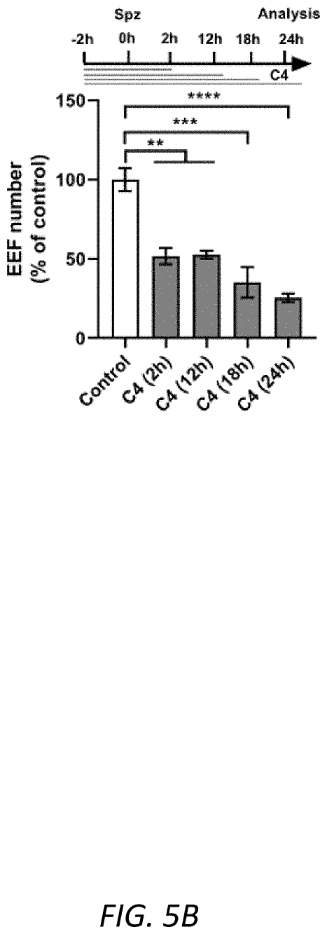

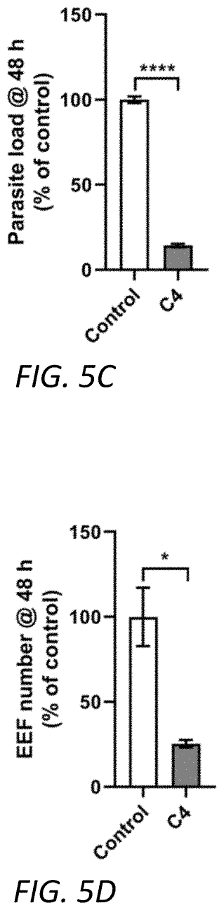

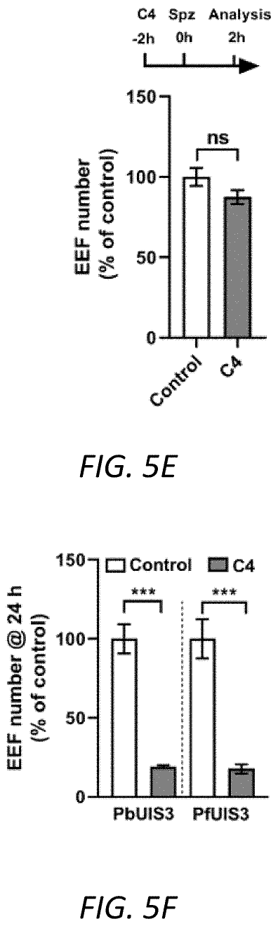

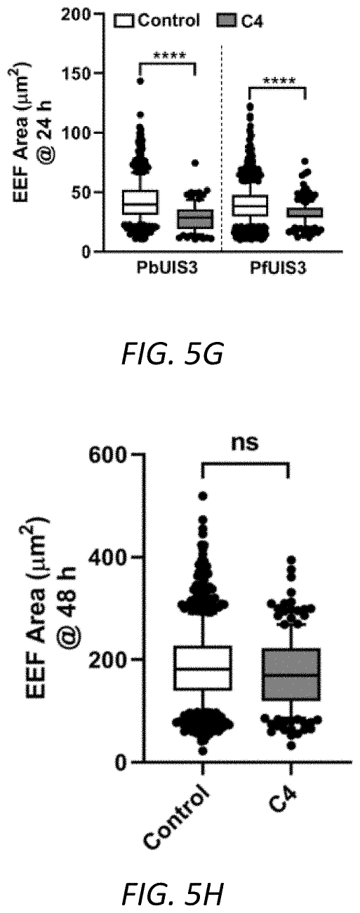

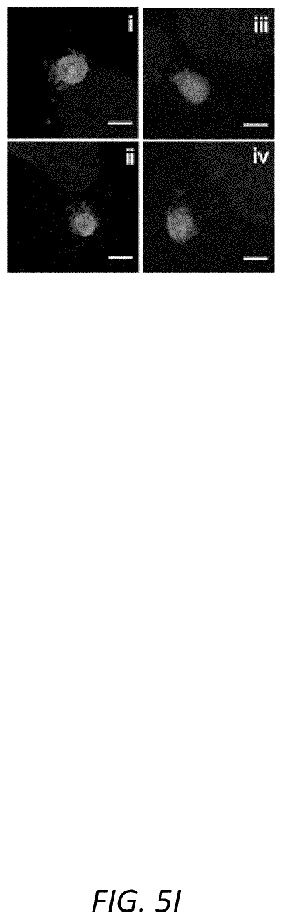

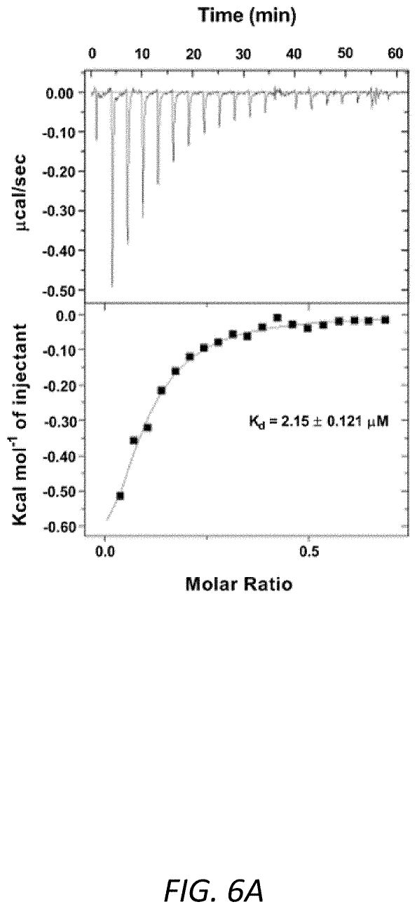

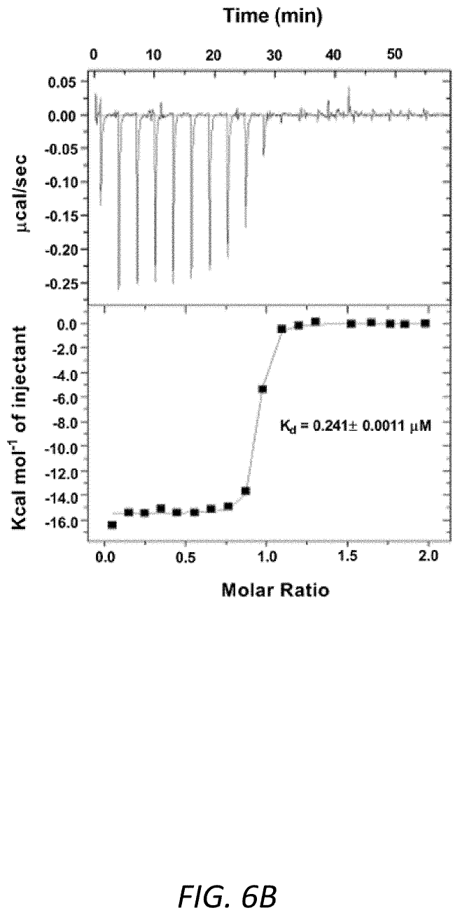

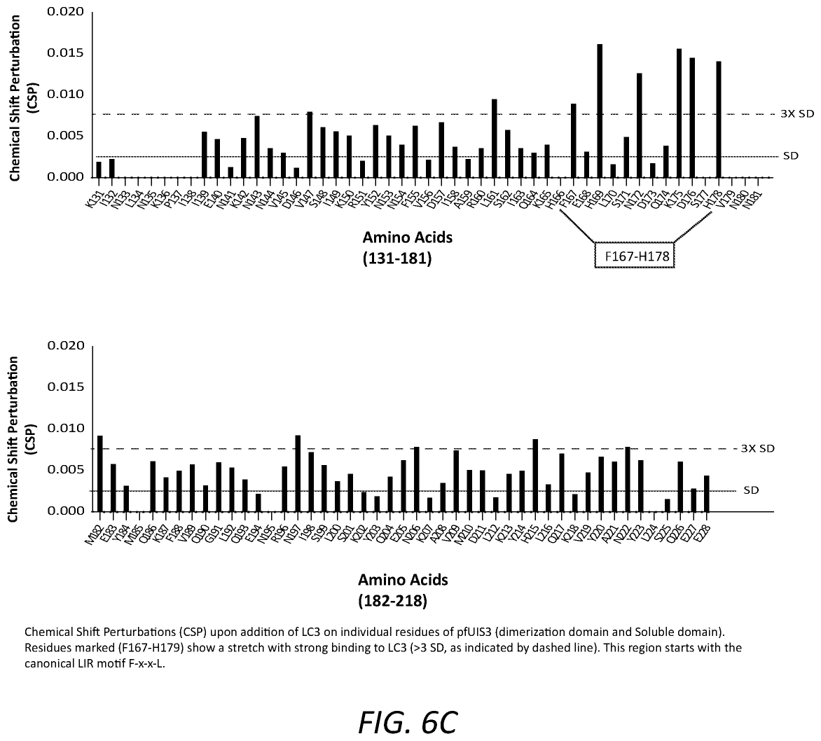

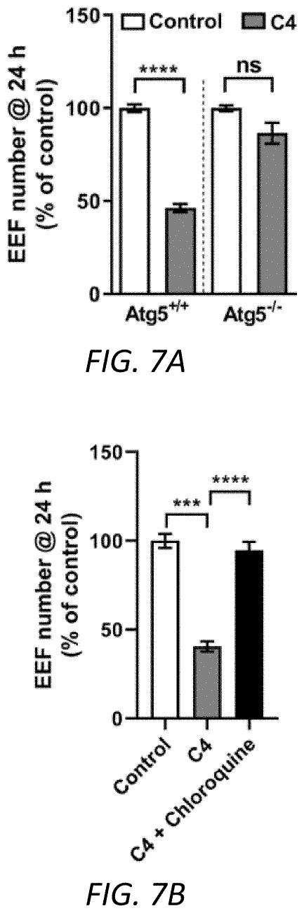

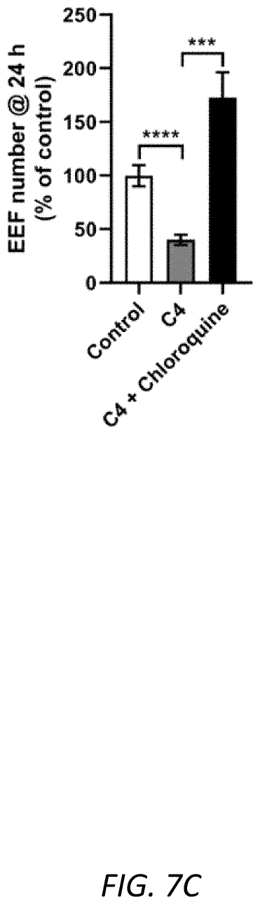

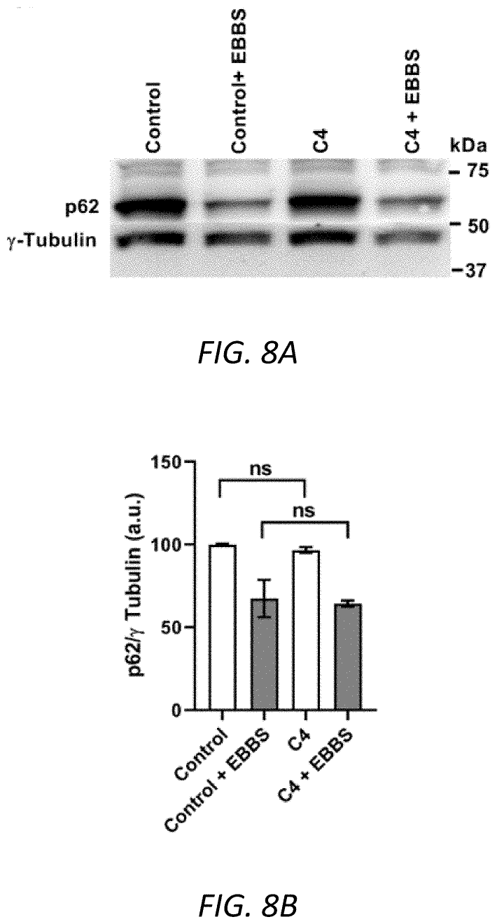

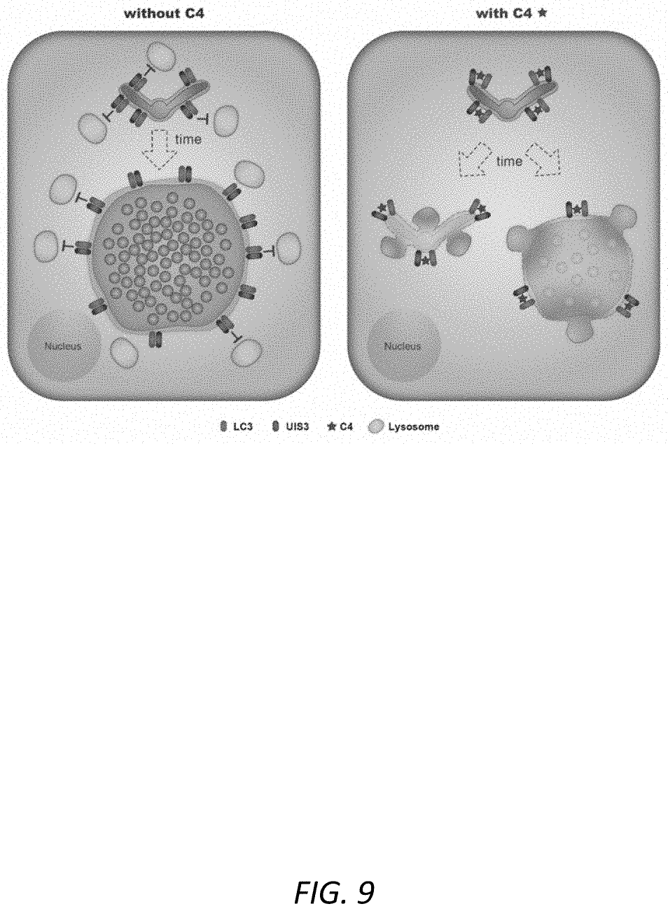

FIGS. 3A-C represent flow cytometry analysis of the antimalarial effect of selected compounds, from the VLS, in P. berghei and PfUIS3@Pb-infected Huh7 cells. Each compound was dissolved in DMSO (0.001%), and added to the cells at a concentration of 10 μM. The cells were infected with P. berghei or PfUIS3@Pb 2 h after the addition of the compounds, and analyzed 24 h post-infection. DMSO (0.001%) was used as a negative control, and Primaquine (10 μM in DMSO (0.001%)) was used as a positive control.

FIG. 3A is a combined bar and line graph showing parasite loads for cells pretreated with compounds C1-C7 and C10-C15, respectively. The bars represent parasite loads and the line represents cell confluency as indicated by the y-axis at the right side of the graph. C4 was identified as the most effective compound for limiting parasite load in case of P. berghei infection. Data represent means±SEM (n=3). P values were calculated using one-way ANOVA with Tukey test, * P<0.1, ** P<0.01, *** P<0.001, **** P<0.0001.

FIG. 3B is a graph showing the number of exo-erythrocytic EEFs, as quantified by the number of infected cells, for cells pretreated with compounds C1-C7, C10-C12, C14, and C15, respectively. The bars represent EEF numbers and the line represents cell confluency as indicated by the y-axis at the right side of the graph. C4 was identified as the most effective compound for limiting the number of EEFs in case of PfUIS3@Pb infection. Data represent means±SEM (n=3). P values were calculated using one-way ANOVA with Tukey test, * P<0.1, ** P<0.01, *** P<0.001, **** P<0.0001.

FIG. 3C is a graph showing the number of exo-erythrocytic forms (“EEF”)s, as quantified by the number of infected cells, for cells pretreated with compound C13 and infected with PfUIS3@Pb.

FIG. 3D shows a ribbon and solvent accessible surface representation of a PbUIS3 homolgy model in complex with docked compound C4 are represented in blue, and soluble domains are represented in in grey. Amino acid residues involved in the interaction of PbUIS3 with LC3 are depicted as sticks, and are identified by position number. The PbUIS3 homology model was built using the crystal structure of PfUIS3 associated with the PDB code 2VWA as reference model.

FIG. 3E shows a ribbon and solvent accessible surface representation of the crystal structure of PfUIS3 associated with the PDB code 2VWA in complex with docked compound C4. Soluble domains are represented in grey and docked compound C4 is represented in blue. Amino acid residues involved in the interaction of PfUIS3 with LC3 are depicted as sticks, and are identified by position number.

FIG. 3F shows a representation of the chemical structure of C4. The ZINC ID No. Of C4 is 25150136, its M.W. is 517.51 g/mol, and its x Log P coefficient is 4.61.

FIG. 4A is a bar graph showing a dose-dependent effect of C4 on the parasite load of P. berghei-infected Huh7 cells, as determined by luciferase assay at 24 h post-infection.

FIG. 4B is a bar graph showing a dose-dependent effect of C4 on the number of EEFs in PfUIS3@Pb-infected Huh7 cells, as determined by flow cytometry analysis at 24 h post-infection.

FIG. 5A is a bar graph showing the effect of 1 μM C4 on the number of EEFs in P. berghei-infected Huh7 cells when the C4 is added either 2 h before infection or at the time of infection. EEFs were quantitated using flow cytometry at 24 h post-sporozoite addition. DMSO (0.0001%) was used as the negative control. Data represent means±SEM (n=3). P values were calculated using one-way ANOVA with Tukey test, ns: non-significant, ** P<0.01, *** P<0.001, **** P<0.0001.

FIG. 5B is a bar graph showing the effect of 1 μM C4 on the number of EEFs in P. berghei-infected Huh7 cells when the C4 is added either 2 h before infection, at the time of infection, or 2, 12, 18 or 24 hours after infection. EEFs were quantitated using flow cytometry at 24 h post-sporozoite addition. DMSO (0.0001%) was used as the negative control. Data represent means±SEM (n=3). P values were calculated using one-way ANOVA with Tukey test, ns: non-significant, ** P<0.01, *** P<0.001, **** P<0.0001.

FIG. 5C is a bar graph showing the parasite load of P. berghei-infected Huh7 cells that were treated with 1 μM C4 2 h prior to infection, as determined by luciferase assay at 48 h post-sporozoite addition. Data represent means±SEM of three replicates of an experiment. Statistical significance was assessed using unpaired two-tailed t-test. ns: non-significant, * P<0.1, **** P<0.0001.

FIG. 5D is a bar graph showing the number of EEFs P. berghei-infected Huh7 cells that were treated with 1 μM C4 2 h prior to infection, as determined by flow cytometic analysis at 48 h post-sporozoite addition. Data represent means±SEM of three replicates of an experiment. Statistical significance was assessed using unpaired two-tailed t-test. ns: non-significant, * P<0.1, **** P<0.0001.

FIG. 5E is a bar graph showing the effect of 1 μM C4 on P. berghei numbers in Huh7 cells as quantified by number of EEFs at 2 h post-infection. C4 was added to the cells 2 h before infection. EEFs were quantitated using flow cytometry. Data represent means±SEM (n=3). Non-parametric two-tailed Mann-Whitney test was used to compare the effect of DMSO control and C4. ns: non-significant.

FIG. 5F is a bar graph showing immunofluorescence-based quantification of the effect of adding 1 μM C4 to the cells 2 h before P. berghei or PfUIS3Pb infection on the number of EEFs. DMSO (0.0001%) was used as the negative control. Data represent means±SEM (n=3). P values were calculated using one-way ANOVA with Tukey test, ns: non-significant, ** P<0.01, *** P<0.001, **** P<0.0001.

FIG. 5G shows EEF sizes in P. berghei and PfUIS3Pb infected Huh7 cells that were treated with 1 μM C4 2 h prior to infection, as determined by mean GFP intensity at 24 h post-sporozoite addition. DMSO (0.0001%) was used as the negative control. Data represent means±SEM (n=3). P values were calculated using one-way ANOVA with Tukey test, ns: non-significant, ** P<0.01, *** P<0.001, **** P<0.0001.

FIG. 5H shows EEF sizes in P. berghei-infected Huh7 cells that were treated with 1 μM C4 2 h prior to infection, as determined by mean GFP intensity at 48 h post-sporozoite addition. Data represent means±SEM of three replicates of an experiment. Statistical significance was assessed using unpaired two-tailed t-test. ns: non-significant, * P<0.1, **** P<0.0001.

FIG. 5I shows representative confocal images of EEFs size comparison (i & ii: P. berghei infection, control and C4; iii & iv: PfUIS3Pb infection, control and C4). Scale bars=5 μm.

FIGS. 6A-6B show that C4 interacts with the UIS3-LC3 complex.

FIG. 6A shows a raw isothermal calorimetry (“ITC”) curve and the ΔH from integration for titration of 100 μM LC3 into 10 μM PfUIS3. (K_{d}=2.15±0.121 μM) Data are representative data of three independent experiment.

FIG. 6B shows a raw ITC curve and the ΔH from integration for titration of 100 μM C4 into a saturation solution of UIS3 with LC3. The data fit a one-site binding model. (K_{d}=0.241±0.0011 μM) Data are representative data of three independent experiment.

FIG. 6C shows Chemical Shift Perturbations (CSP) upon addition of LC3 on individual residues of pfUIS3 (dimerization domain and Soluble domain). Residues marked (F167-H178) show a stretch with strong binding to LC3 (>3 SD, as indicated by dashed line). This region starts with the canonical LIR motif F-x-x-L.

FIGS. 7A-7C show that C4 employs host autophagy pathway for its anti-plasmodial effect leading to parasite elimination from the host cells.

FIG. 7A shows the effect of 10 μM C4 on P. berghei infection in control (Atg5^{+/+}) and autophagy-deficient (Atg5^{-/-}) mouse embryonic fibroblasts (“MEFs”). Data represent means±SEM (n=3). Statistical significance was assessed using the unpaired two-tailed t-test. ns: non-significant, **** P<0.0001.

FIG. 7B shows the anti-parasitic effect of 1 μM C4 on P. berghei infection in Huh7 cells can be reverted using the known autophagy inhibitor Chloroquine (50 μM). C4 and Chloroquine were added to the cells 2 h before infection. The effect was analysed at 24 h post-infection using flow cytometry by quantifying the EEFs number in infected cells. Data represent means±SEM. Pooled data from 3 independent experiments are presented. Statistical significance was assessed using the unpaired two-tailed t-test. *** P<0.001, **** P<0.0001.

FIG. 7C shows the anti-parasitic effect of 1 μM C4 on PfUIS3@Pb infection in Huh7 cells can be reverted using the known autophagy inhibitor Chloroquine (50 μM). C4 and Chloroquine were added to the cells 2 h before infection. The effect was analysed at 24 h post-infection using flow cytometry by quantifying the EEFs number in infected cells. Data represent means±SEM. Pooled data from 4 independent experiments are presented. Statistical significance was assessed using the unpaired two-tailed t-test. *** P<0.001, **** P<0.0001.

FIGS. 8A-8B show that C4 does not obstruct the intrinsic autophagy flux of the host cell.

FIG. 8A shows an immunoblot of p62 levels in HeLa cells following treatment with either 1 μM C4 or 0.0001% DMSO (control) for 24 h, and then changed from normal growth medium to EBBS to induce amino-acid starvation dependent autophagy Results are representative of two independent experiments.

FIG. 8B is a bar graph showing the p62 levels before and after amino acid starvation were calculated as the ratio of p62 to y tubulin (loading control). Data represent means±SEM (n=2). Statistical significance was assessed using the unpaired two-tailed t-test. ns: non-significant.

FIG. 9 is a depiction of a proposed mode of action for the anti-malarial small molecule C4 on Plasmodium liver-stage infection. Data, described herein, support a model in which C4 overcomes the protective function of UIS3 during Plasmodium intrahepatic development. In the presence of C4, UIS3 cannot sequester LC3 rendering the downstream autophagy receptors to interact with it. Consequently, the host autophagy pathway remains active leading to the lysosome fusion with the PVM and elimination of the parasite from the hepatocyte.

### DETAILED DESCRIPTION

This disclosure describes compositions and methods for treating Plasmodium infections by using small molecule compounds to disrupt direct interactions between a host cell's autophagosome membrane-associated protein, microtubule-associated protein 1 light chain 3 (“LC3”), and the parasitophorous vacuole membrane (“PVM”)-associated protein, Upregulated in Infective Sporozoites 3 (“UIS3”). Accordingly, compositions of the invention include inhibitors of the interaction between host cell LC3 and parasite UIS3—typically an inhibitor that prevents direct binding between the proteins. Such inhibitors of the invention may be, for example, small molecule drugs that prevent the sequestration of host cell LC3 by Plasmodium UIS3 on the surfaces of PVMs, thereby allowing the host cells to eliminate the parasite by an autophagy-dependent process.

Some small molecule drugs of the invention are phenyloxadiazole compounds that inhibit LC3 sequestration by UIS3. In some compositions and methods of the invention, the small molecule drug contains an oxadiazole ring connected to a tri-fluoro-methyl-benzene and an N-alkyl-piperazine, linked to a nitrile derivative of benzoic acid. In certain methods and compositions of the invention, the small molecule drug is (4-{[4-(4-{5-[3-(trifluoromethyl) phenyl]-1,2,4-oxadiazol-3-yl}benzyl)piperazino]carbonyl}benzonitrile, which is referred to here as “C4”.

The direct interaction between LC3 and UIS3 disrupted by a small molecule drug of the invention may involve one or more of an ionic interaction, hydrogen bond, or a Van der Waals interaction between at least one amino acid of UIS3 and at least one amino acid of LC3, including, for example, interactions between certain atoms or functional groups of the side-chains. Therefore, the disruption of the direct interaction between UIS3 and LC3 by a small molecule drug of the invention may be the result of drug's blocking and/or interference of binding between UIS3 and LC3 at specified amino acid positions. In some methods and compositions of the invention, a small molecule drug blocks and/or interferes with UIS3's interaction with LC3 at one or more of the following UIS3 amino acids: N181, E183, M182, K213, and Q217 of P. falciparum UIS3, for example, the P. falciparum UIS3 sequence defined in SEQ. ID. NO. 1. In the same, or other methods and compositions of the invention, a small molecule drug blocks and/or interferes with UIS3's interaction with LC3 at one or more amino acids of a Plasmodium UIS3 protein positions 167-178, for example at F167-H178.

In some methods and compositions of the invention, the small molecule drug binds a UIS3-LC3 complex with a higher affinity than for the affinity of the direct binding of UIS3 and LC3. Binding affinity can be measured by the dissociation rate between two molecules. Various methods can be used to measure binding affinity, including, for example, methods that utilize surface plasmon resonance (SPR), competitive radioimmunoassay, ELISA, and isothermal calorimetry. In certain methods and compositions of the invention, the small molecule drug binds a complex of UIS3 and LC3 with at least a 2 fold, at least a 4 fold, at least 6 fold, at least 8 fold, at least 10 fold, at least 12 fold, at least 14 fold, at least 16 fold, at least 18 fold, at least 20 fold, at least 100 fold, at least 1000 fold, or at least 1×10⁴ fold greater than the affinity of the interaction between UIS3 and LC3. For example, in some methods and compositions of the invention, C4 binds a UIS3-LC3 complex with at least 5-fold, at least 6-fold, at least 7-fold, at least 8-fold, at least 9-fold, or at least 10 fold higher affinity than for the affinity of the direct binding of UIS3 and LC3.

As discussed above, compositions and methods of the invention are useful for treating Plasmodium infections in subjects in need thereof. Therefore, as Plasmodium parasites are the organisms associated with malarial diseases, methods and compositions of the invention can be used to treat Malaria. Accordingly, a “subject in need thereof”, as used herein, refers to a human or non-human animal, that has contacted, or thought to have contacted, a Plasmodium parasite infection. “Treatment” or “treating” as used herein means curative or therapeutic treatment to restoring health. Thus, a “treatment”, “method of treatment”, “therapy”, of the invention, facilitates elimination of Plasmodium parasites from Plasmodium—infected cells of the subject, thereby preventing the further advancement of the infection and malarial symptoms. In some methods of the invention, treatment of a Plasmodium infection facilitates liver stage Plasmodium parasites elimination from host cells.

A therapeutically effective amount of an inhibitor of the invention—typically a small molecule drug—is a dosage amount that is sufficient to facilitate elimination of Plasmodium parasites from infected cells of a subject. The dosage amount will depend upon a number of factors, including biological activity, mode of administration, frequency of treatment, type of concurrent treatment, if any, age, body weight, sex, general health, severity of the Plasmodium infection to be treated, as well as appropriate pharmacokinetic properties. Compositions and methods according to the invention are useful for treating an infection by any Plasmodium species. Accordingly, in some methods and compositions of the invention, the small molecule drug facilitate elimination of parasites from a subject suffering from a P. falciparum infection, a P. vivax infection, a P. ovale infection, a P. malariae infection, or a P. knowlesi infection.

Methods and compositions of the invention may also combine a small molecule drug that disrupts the direct interaction of UIS3 and LC3 with one or more antimalarial drugs, either in a single pharmaceutical composition, or in separate, respective pharmaceutical compositions, which are administered to a subject in need thereof either separately or simultaneously. For example, in some compositions and methods of the invention, the small molecule drug inhibitor of the UIS3-LC3 complex is coadministered with: an AMPK activation agent, Quinine or a quinine-related agents; Chloroquine; Amodiaquine; Pyrimethamine; Atovaquone; Artemisinin or a artemisinin derivative; Halofantrine; Doxycycline; Clindamycin; 8 aminoquinoline or an 8 aminoquinoline derivative drug; a Dipeptidyl peptidase-4 (“DPP-4”) inhibitor; or any combination thereof.

AMPK activation agents, include, but are not limited to: Thienopyridone derivatives (e.g., compounds described in WO 2009135580, WO 2009124636, US 20080221088, and EP 1754483); Imidazole derivatives (e.g., compounds described in WO 2008120797 and EP 2040702); Thiazole derivatives (e.g., compounds described in EP1907369); 2-deoxy-D-glucose; compound A769662, Guanidine and its derivatives; Biguanides (e.g. synthalin, PS-15, chlorproguanil, proguanil, buformin, phenformin, and metformin); Natural product-derived agents, such as thiazolidinones (e.g. ciglitazone, MCC-555, rivoglitazone, troglitazone, rosiglitazone, and pioglitazone), adiponectin, Ciliary Neurotrophic Factor (CNTF), ghrelin, salicylate, alpha-lipoic acid, alkaloids, and bitter melon extracts; Plant polyphenols such as, resveratrol, nootkatone, cucurbitane triterpenoid, momordicoside A, nectandrin B, obovatol, glabridin, damulin B, quercetin, ginsenoside, curcumin, berberine, epigallocatechin gallate, theaflavine, hispidulin. A “biguanide drug”, or “biguanide”, has a base chemical structure of C2H7N5. Biguanide class drugs decrease hepatic glucose production and intestinal absorption of glucose in subjects. Exemplary biguanides of the invention include synthalin, PS-15, chlorproguanil, proguanil, phenformin, buformin, phenformin, and metformin, as well as salts of biguanides, such as hydrochloride, acetate, maleate, fumarate, and succinate salts.

Inhibitors of the direct interaction between host cell LC3 and parasite UIS3 according to the invention may be formulated for administration to a subject in need thereof. Formulations of the invention may be single pharmaceutical compositions or dosage forms or in separate pharmaceutical compositions. A “pharmaceutical composition” refers to a solid or liquid composition, which includes at least one inhibitor of the invention, as described above, and at least one carrier, diluent, or excipient. For example, in a solid dosage form of a pharmaceutical compositions of the invention, at least one inhibitor of the invention combined with at least one pharmaceutically acceptable excipient such as a: (a) filler or extender, such as, a starch, lactose, sucrose, glucose, mannitol, and silicic acid; (b) binder, such as a cellulose derivative, a starch, an aliginate, gelatin, polyvinylpyrrolidone, sucrose, and gum acacia; (c) humectant, such as glycerol; (d) disintegrating agent, such as agar-agar, calcium carbonate, potato or tapioca starch, alginic acid, croscarmellose sodium, complex silicates, and sodium carbonate; (e) solution retarder, such as paraffin; (f) absorption accelerator, such as a quaternary ammonium compound; (g) wetting agent, such as cetyl alcohol, glycerol monostearate, and magnesium stearate; (h) adsorbent, such as kaolin and bentonite; and (i) lubricant, such as talc, calcium stearate, magnesium stearate, solid polyethylene glycols, sodium lauryl sulfate.

Solid dosage forms of pharmaceutical compositions according to the invention, such as oral dosage forms, can be prepared as a capsules or tablets, and may have a coating or shells, such as enteric coating. Oral dosage forms may also be prepared to release an inhibitor of the invention at one or more specified locations of the intestinal tract.

A pharmaceutical composition or dosage form of the invention can also be in the form of a suspension, which, in addition to containing an inhibitor of the invention may also contain one or more: (a) suspending agents, such as ethoxylated isostearyl alcohol, polyoxyethylene sorbitol and sorbitan esters, microcrystalline cellulose, aluminum metahydroxide, bentonite, agar-agar and tragacanth; (b) a pharmaceutically acceptable solvent; (c) a buffering agent; (d) a flavorant; (e) a sweetening agent; (f) a preservative; and (g) a stabilizing agent.

Pharmaceutical compositions or dosage forms of the invention can also be prepared for parenteral administration. For example, in various embodiments, at an inhibitor of the invention is included in a composition with injectable pharmaceutically and physiologically acceptable fluids such as water, physiological saline, balanced salt solutions, aqueous dextrose, or glycerol as a vehicle.

It is also possible to provide pharmaceutical compositions or dosage forms of the invention onto a dermal patch to generate a transdermal delivery apparatus and applying such a patch onto the skin in order to attain effective superficial treatment or enhanced penetration of an inhibitor of the invention into the skin or through the skin. Therefore, in certain embodiments of the invention, at least one an inhibitor of the invention is delivered to the skin using conventional dermal-type patches or articles, wherein the inhibitor of the invention is contained within a laminated structure that serves as a drug delivery device to be affixed to the skin.

Pharmaceutical compositions according to the invention can also be included in kits. Such kits can be in the form of a closed package system, containing at least one inhibitor of the invention. When supplied as a kit, the pharmaceutical composition or compositions can be provided in separate containers, which can, optionally, be packaged under a neutral non-reacting gas, such as nitrogen. The containers may be composed of any suitable material, such as glass, organic polymers, such as polycarbonate, polystyrene, ceramic, metal or any other material typically employed to hold pharmaceutical compositions. Containers may have a sterile access port, such as a bottle having a stopper that can be pierced by a hypodermic injection needle, which, optionally, may also be included in a kit. Other containers may have two compartments that are separated by a readily removable membrane that upon removal permits the components, such as an inhibitor of the invention to mix. Removable membranes may be glass, plastic, rubber, and the like. In certain embodiments, kits can also be supplied with instructional materials. Instructions may be printed on paper or other substrate, or may be supplied as an electronic-readable medium. Detailed instructions may not be physically associated with the kit; instead, a user may be directed to an Internet web site specified by the manufacturer or distributor of the kit.

### EXAMPLES

The following examples exemplify an efficient anti-malarial strategy by proving that the disruption of a critical host-parasite interaction, without affecting normal host function, disrupts the first and obligatory step of Plasmodium parasites infection. See FIG. 9 for an overview of an anti-malarial strategy supported by the examples, below.

### Example 1. Identification of the Small Molecule C4 Hampering Host Cell Infection by Plasmodium sporozoites

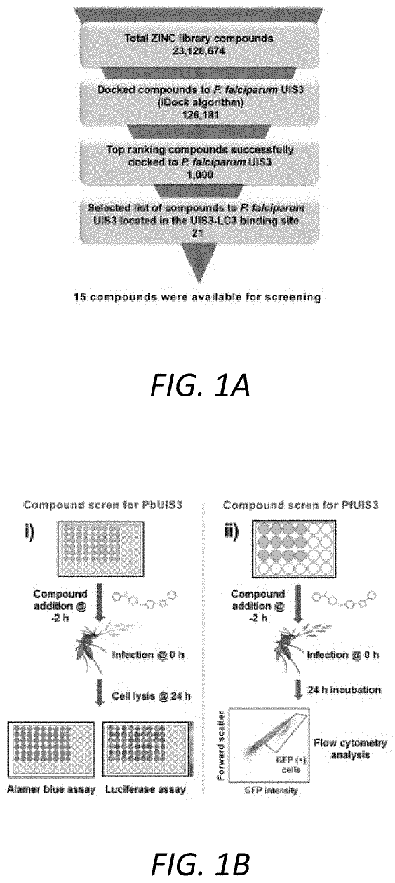

To identify a small molecule compound or compounds with the potential to disrupt binding of UIS3 to LC3, a virtual compound library screen (“VLS”) of more than 20 million compounds contained in the open-access library, ZINC chemical compound database, (Department of Pharmaceutical Chemistry at the University of California, San Francisco) was performed using an in silico molecular docking analysis of P. falciparum UIS3 and human LC3 to identify compounds with the potential to bind at the UIS3-LC3 interacting region. The docking analysis was based on published X-ray structures of a complex of P. falciparum UIS3 and human LC3 (Real et al.). The published X ray structures were for uncomplexed UIS3 (PDB code: 2VWA) and LC3 (PDB code: 2ZJD). See FIG. 1A for a schematic on the VLS strategy. The screening of inhibitors of UIS3-LC3 binding was performed by the iDock algorithm (Li et al.).

A cubic searching space of 30×30×30 Å, centered at the LC3-binding pockets located in the surface of UIS3 protein from P. berghei and P. falciparum, were used as a starting point for the virtual screening. Among the 23,128,674 ligands in ZINC database, 126,181 were successfully docked to UIS3 surface pockets. From the 1,000 top ranked ligands sorted by the Gibb's function, the best 21 docking solutions involving the UIS3 residues previously characterized as important for LC3 binding (Real et al.), were further refined by energy minimization using a knowledge-based potential of mean force and stereochemistry correction with GROMACS (Pronk, S. et al). Of the 21 selected compounds, 15 were available for use. These 15 compounds are listed in Table 1.

TABLE 1
Compounds with potential to disrupt UIS3 binding to LC3

| Compound | ZINC ID | Structure | |
|---|---|---|---|
| C1 | 15243482 | 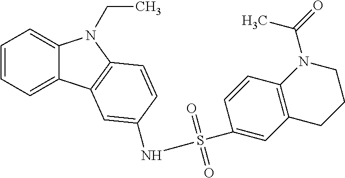 [SMILES: CCn1c2ccccc2c2cc(NS(=O)(=O)c3ccc4c(c3)CCCN4C(C)=O)ccc21] | |
| | | MW (g/mol) | 447.56 |
| | | xlogP | 4.60 |
| | | Rotatable Bonds | 4 |
| | | Polar surface area (Å²) | 71 |
| C2 | 873264 | 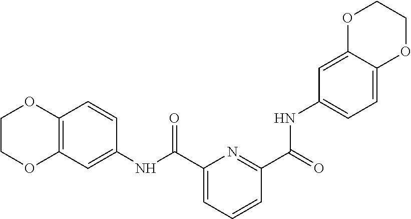 [SMILES: O=C(Nc1ccc2c(c1)OCCO2)c1cccc(C(=O)Nc2ccc3c(c2)OCCO3)n1] | |
| | | MW (g/mol) | 433.42 |
| | | xlogP | 1.64 |
| | | Rotatable Bonds | 4 |
| | | Polar surface area (Å²) | 108 |
| C3 | 7599237 | 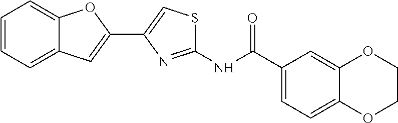 [SMILES: O=C(Nc1nc(-c2cc3ccccc3o2)cs1)c1ccc2c(c1)OCCO2] | |
| | | MW (g/mol) | 378.40 |
| | | xlogP | 3.51 |
| | | Rotatable Bonds | 3 |
| | | Polar surface area (Å²) | 73 |
| C4 | 25150136 | 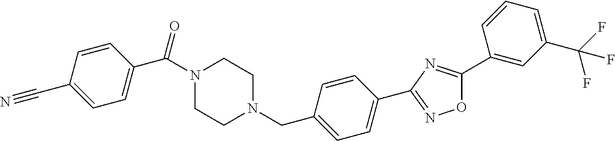 [SMILES: N#Cc1ccc(C(=O)N2CCN(Cc3ccc(-c4noc(-c5cccc(C(F)(F)F)c5)n4)cc3)CC2)cc1] | |
| | | MW (g/mol) | 517.51 |
| | | xlogP | 4.61 |
| | | Rotatable Bonds | 6 |
| | | Polar surface area (Å²) | 86 |
| C5 | 14242199 | 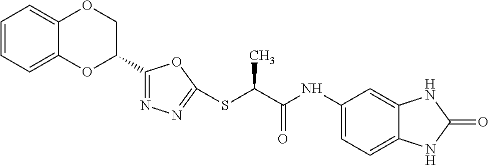 [SMILES: C[C@H](Sc1nnc([C@H]2COc3ccccc3O2)o1)C(=O)Nc1ccc2[nH]c(=O)[nH]c2c1] | |
| | | MW (g/mol) | 439.45 |
| | | xlogP | 1.56 |
| | | Rotatable Bonds | 5 |
| | | Polar surface area (Å²) | 135 |
| C6 | 35399629 | 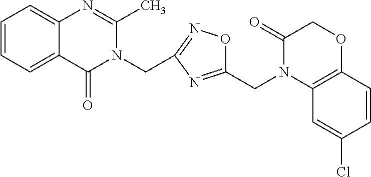 [SMILES: Cc1nc2ccccc2c(=O)n1Cc1noc(CN2C(=O)COc3ccc(Cl)cc32)n1] | |
| | | MW (g/mol) | 437.84 |
| | | xlogP | 2.18 |
| | | Rotatable Bonds | 4 |
| | | Polar surface area (Å²) | 103 |
| C7 | 9419593 | 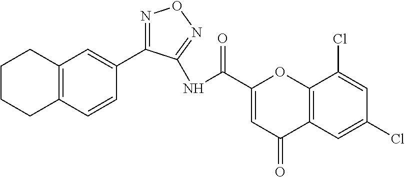 [SMILES: O=C(Nc1nonc1-c1ccc2c(c1)CCCC2)c1cc(=O)c2cc(Cl)cc(Cl)c2o1] | |
| | | MW (g/mol) | 456.28 |
| | | xlogP | 5.65 |
| | | Rotatable Bonds | 3 |
| | | Polar surface area (Å²) | 98 |
| C8 | 36076978 | 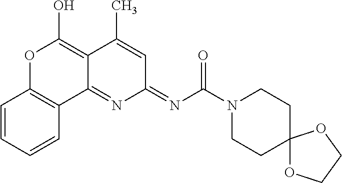 [SMILES: Cc1c/c(=N\C(=O)N2CCC3(CC2)OCCO3)nc2c3ccccc3oc(O)c1-2] | |
| | | MW (g/mol) | 411.45 |
| | | xlogP | 3.07 |
| | | Rotatable Bonds | 2 |
| | | Polar surface area (Å²) | 100 |
| C9 | 32999120 | 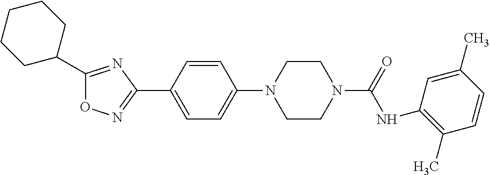 [SMILES: Cc1ccc(C)c(NC(=O)N2CCN(c3ccc(-c4noc(C5CCCCC5)n4)cc3)CC2)c1] | |
| | | MW (g/mol) | 459.59 |
| | | xlogP | 6.10 |
| | | Rotatable Bonds | 4 |
| | | Polar surface area (Å²) | 74 |
| C10 | 10994401 | 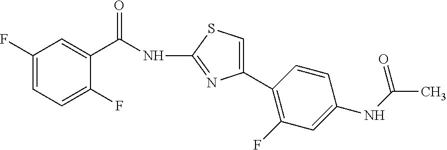 [SMILES: CC(=O)Nc1ccc(-c2csc(NC(=O)c3cc(F)ccc3F)n2)c(F)c1] | |
| | | MW (g/mol) | 391.37 |
| | | xlogP | 3.12 |
| | | Rotatable Bonds | 4 |
| | | Polar surface area (Å²) | 71 |
| C11 | 72128248 | 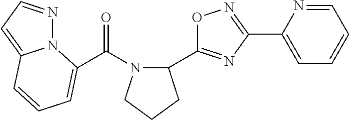 [SMILES: O=C(c1cccc2ccnn12)N1CCCC1c1nc(-c2ccccn2)no1] | |
| | | MW (g/mol) | 360.37 |
| | | xlogP | 1.74 |
| | | Rotatable Bonds | 3 |
| | | Polar surface area (Å²) | 89 |
| C12 | 20610177 | 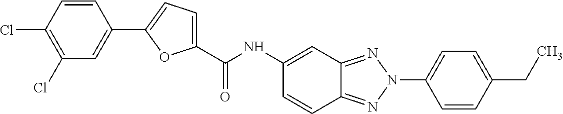 [SMILES: CCc1ccc(-n2nc3ccc(NC(=O)c4ccc(-c5ccc(Cl)c(Cl)c5)o4)cc3n2)cc1] | |
| | | MW (g/mol) | 477.35 |
| | | xlogP | 6.87 |
| | | Rotatable Bonds | 5 |
| | | Polar surface area (Å²) | 73 |
| C13 | 19852631 | 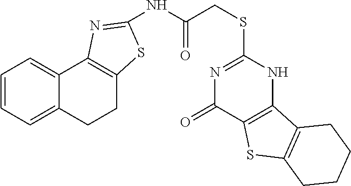 [SMILES: O=C(CSc1nc(=O)c2sc3c(c2[nH]1)CCCC3)Nc1nc2c(s1)CCc1ccccc1-2] | |
| | | MW (g/mol) | 480.64 |
| | | xlogP | 5.34 |
| | | Rotatable Bonds | 4 |
| | | Polar surface area (Å²) | 88 |
| C14 | 66982809 | 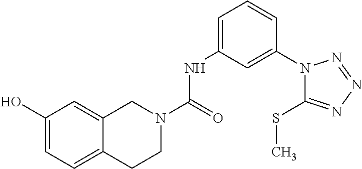 [SMILES: CSc1nnnn1-c1cccc(NC(=O)N2CCc3ccc(O)cc3C2)c1] | |
| | | MW (g/mol) | 382.44 |
| | | xlogP | 2.34 |
| | | Rotatable Bonds | 3 |
| | | Polar surface area (Å²) | 96 |
| C15 | 64193563 | 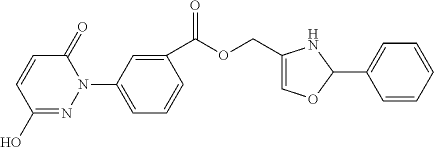 [SMILES: O=C(OCC1=COC(c2ccccc2)N1)c1cccc(-n2nc(O)ccc2=O)c1] | |
| | | MW (g/mol) | 388.35 |
| | | xlogP | 3.72 |
| | | Rotatable Bonds | 6 |
| | | Polar surface area (Å²) | 110 |

### Example 2. Phenotypic Screen of Compounds Selected in the VLS

The selected compounds from the VLS were next tested in a phenotypic screen (“PHS”, FIG. 1B), with the exceptions of compounds 8 and 9, which were excluded from the screening due to their insolubilities in DMSO.

The following two Plasmodium parasite strains were used in the PHS: i) the rodent wild-type (WT) GFP expressing P. berghei ANKA parasite strain (259cl2, obtained from the Leiden Malaria Research Group); and ii) the parasite line, PfUIS3@Pb, created by inserting the gene encoding P. falciparum UIS3 with an added a 2 HA tag (UIS3-HA) into the P. berghei parasite (507cl1, obtained from the Leiden Malaria Research Group) at the UIS3 locus under the control of native 5′ and 3′ regulatory sequences. See FIG. 1C. More particularly, to replace the P. berghei UIS3 open reading frame (ORF) with UIS3 ORF of P. falciparum, a double crossover recombination was performed as described in FIG. 2A. Transfection was carried out by electroporation of purified schizonts as previously described. (Janse et al.) Parasites were maintained under Pyrimethamine selection pressure, collected on day 10 post-transfection and genotyped using a PCR-based method with the primers listed in Table 3. Genotyping of PfUIS3@Pb confirmed the correct integration of the PfUIS3-HA expression cassette. See FIG. 2B.

P. berghei sporozoites used in these studies were obtained through dissection of the salivary glands of infected female Anopheles stephensi mosquitoes bred at the Instituto de Medicina Molecular.

TABLE 2
Primers used to generate the P. falciparum
UIS3-HA transfection construct.

| | Primer pairs |
|---|---|
| PCR Product | (Forward; Reverse) |
| PfUIS3-HA | 5′ ATGGGCCCATGAAGGTCT |
| | CTAAATTAGTCTTG 3′ |
| | (SEQ ID NO. 2) |
| | 5′ ATGCGGCCGCTTATGCAT |
| | AATCTGGTACATCATATGGAT |
| | ATGCATAATCTGGTACATCAT |
| | ATGGATAGTTCTCTTCTTGAG |
| | ATAAATAATTAG 3′ |
| | (SEQ ID NO. 3) |
| PbUIS3 | 5′ TAAAGCTTAAGGATTATA |
| 5′ UTR | TTTTATAATGTTTCAC 3′ |
| | (SEQ ID NO. 4) |
| | 5′ ATGGGCCCTTTTATACAC |
| | TTTCATATATTTGTTAT 3′ |
| | (SEQ ID NO. 5) |
| PbUIS3 | 5′ TAGGTACCATGTTTGTG |
| 3′ UTR | TAACATCATTTATAG 3′ |
| | (SEQ ID NO. 6) |
| | 5′ ATAAGCTTTTCATATAT |
| | CAATTTTCAAATTG 3′ |
| | (SEQ ID NO. 7) |
| PbUIS4 | 5′ ATGCGGCCGCTTCATTA |
| 3′ UTR | TGAGTAGTGTAATTCAG 3′ |
| | (SEQ ID NO. 8) |
| | 5′ ATGAATTCGCATACAAC |
| | ATATGTAAAAAAG 3′ |
| | (SEQ ID NO. 9) |

TABLE 3
Primers used for PfUIS3@Pb genotyping

| | Primer pairs |
|---|---|
| PCR Product | (Forward; Reverse) |
| Recombinant | 5′ ATTATATTGTGCTATAAAGCG 3′ |
| locus 5′ | (SEQ ID NO. 10) |
| integration | 5′ GCATTTAAACAAATATATTGAC |
| | ATAAGAT 3′ (SEQ ID NO. 11) |
| WT locus | 5′ TAAAGCTTAAGGATTATATTTT |
| | ATAATGTTTCAC 3′ |
| | (SEQ ID NO. 12) |
| | 5′ GCAGCTAGTTTCACATTATCCA |
| | TAAATAT 3′ |
| | (SEQ ID NO. 13) |
| Control | 5′ TAAAGCTTAAGGATTATATTTT |
| (PbUIS3 | ATAATGTTTCAC 3′ |
| 5′ UTR) | (SEQ ID NO. 14) |
| | 5′ ATGGGCCCTTTTATACACTTTC |
| | ATATATTTGTTAT 3′ |
| | (SEQ ID NO. 15) |

Immunofluorescence microscopy analysis of PfUIS3@Pb-infected Huh7 cells showed co-localization of PfUIS3-HA with another known PVM marker protein, UIS4, indicating the correct localization of the PfUIS3-HA at the PVM. See FIG. 2C. WT and PfUIS3@Pb parasite lines behaved similarly throughout the parasite life cycle, including with respect to: i) the number of salivary gland sporozoites each line produced, which was an average of 30,000 sporozoites per mosquito; ii) liver-stage infectivity (FIGS. 2D-E); and iii) blood parasitaemia (FIG. 2G); and iv) disease progression (FIG. 2H). PVM localization of PfUIS3-HA and liver stage development of PfUIS3@Pb parasites (EEF number and size comparison with WT) was performed as described previously.

To assess C4 effect on EEF number and size, Huh7 cells were plated on glass coverslips (40,000/cover slip), treated with 1 μM C4 or 0.0001% DMSO (control) and after 2 h infected with P. berghei/PfUIS3@Pb sporozoites (40,000/cover slip). Huh7 cells (obtained from ATCC) were cultured under standard conditions in RPMI 1640 and DMEM medium (Gibco Invitrogen) respectively supplemented with 10% FCS, 1% glutamine, 1% non-essential amino acids (RPMI 1640), 1% penicillin/streptomycin, and 1% Hepes (RPMI 1640).

To characterize PfUIS3@Pb parasite line, Huh7 cells were seeded on cover slips as above and infected with WT (P. berghei ANKA (507cl1)/PfUIS3@Pb sporozoites (40,000/cover slip). After 24 h, cells were fixed in 4% paraformaldehyde (ChemCruz) for 15 min at room temperature (RT), permeabilized/blocked (PBS, 0.1% Triton X-100, 1% BSA) for 1 h at RT and incubated with primary antibodies (diluted in blocking solution) for overnight at 4° C. Cells were then washed with PBS, incubated with AlexaFluor-conjugated secondary antibodies (Jackson ImmunoResearch Laboratories) and Hoechst 33342 (Invitrogen) for 1 h at RT and washed again. The coverslips were mounted with Fluoromount (SouthernBiotech). The following primary antibodies were used: PbUIS4 (goat polyclonal, SicGen, AB0042-200, 1:1000) and HA (mouse monoclonal, BioLegend, 901509, 1:500). GFP signal was detected using AlexaFluor 488 conjugated anti GFP (rabbit polyclonal, Invitrogen, A-21311, 1:500) antibody. For infection quantification by microscopy, images (36 per cover slip) were acquired on a Zeiss Axiovert 200M wide-field microscope equipped with an automated stage. In other cases, images were acquired on a Zeiss confocal microscopes (LSM 710/LSM 880). All images were processed and analysed using ImageJ.

The PHS was performed using Huh7 cells infected with sporozoites from either parasite lines with or without adding each of the compounds, at a concentration of 10 μM, 2 h before sporozoite addition. Infection level was determined either by measuring luminescent levels of the GFP and firefly luciferase-expressing P. berghei ANKA (676m1cl1), (obtained from the Leiden Malaria Research Group) or the number of GFP-positive infected cells by flow cytometry, representing Plasmodium exo-erythrocytic form, EEF, (as PfUIS3@Pb parasite line express GFP).

To perform the flow cytometry analysis, Huh7 cells were seeded on a 24 well plate (50,000 cells/well) in RPMI and DMEM media respectively. The next day, media was replaced with different compounds (10 μM)/C4 (various conc.)/1 μM C4/1 μM C4+50 μM Chloroquine containing media, as appropriate in different experiments. Cells were then infected with P. bergehi/PfUIS3@Pb parasites and analysed by flow cytometry at 2 h/24 h post infection. DMSO was used as control and Primaquine was used as the positive control. For flow cytometry, infected cells were trypsinized and resuspended in 300 μl of media. 50 μl of each sample were analysed on BD Accuri C6 as described in Hanson et al. Data were analysed using FlowJo software.

Toxicity of the compounds towards the host cell was determined by measuring host cell confluency using Alamar blue assay (Promega) (FIG. 3A) and flow cytometry analysis (FIGS. 3B-C). In both screens for the two independent parasite lines, the compound C4 ((4-{[4-(4-{5-[3-(trifluoromethyl) phenyl]-1,2,4-oxadiazol-3-yl}benzyl)piperazino]carbonyl}benzonitrile) was identified as the best hit, responsible for the highest decrease in infection (FIGS. 3A-C).

C4 binds in a pocket that partially overlaps the proposed UIS3-LC3 interacting region for both P. falciparum and P. berghei UIS3 (FIGS. 3D-E). C4 belongs to the class of phenyloxadiazoles, which are polycyclic aromatic compounds containing a benzene ring linked to a 1,2,4-oxadiazole ring through a C—C or C—N bond. The oxadiazole ring is connected to a tri-fluoro-methyl-benzene and an N-alkyl-piperazine, linked to a nitrile derivative of benzoic acid (FIG. 3F).

### Example 3. C4 Impacts Parasite Survival Inside the Hepatocytes Cells

An optimal working concentration of C4 that can significantly decrease infection with both P. berghei ANKA and PfUIS3@Pb parasite lines was determined as described below. First, it was demonstrated that C4 decreased infection in cells infected with either P. berghei ANKA or PfUIS3@Pb in a dose-dependent manner. The IC₅₀ for C4 in P. berghei ANKA-infected and PfUIS3@Pb-infected cells at 24 h. Post-infection was 93.3 nM and 188.2 nM, respectively. See FIGS. 4A and 4B. To analyze C4 dose-dependent effect, media were replaced with DMSO (0.0001%)/C4 (various conc.)-containing media. After 2 h, cells were infected with luciferase-expressing P. berghei sporozoites (10,000/well) and processed for Luciferase assay at 24 h post-infection. Flow cytometry was used to determine EEF counts.

When C4 was used in this infection model at a concentration of 1 μM, it decreased infection from a very early time-point post-sporozoite addition. That effect increased with longer incubations up to 24 h post-infection, and was maintained through at least 48 h post-infection. See FIGS. 5A-D. C4 did not affect Plasmodium sporozoite invasion of host cells. See FIG. 5E. Microscopic analysis of both P. berghei ANKA and PfUIS3@Pb parasite-infected Huh7 cells confirmed that C4 impacted parasite infection by causing a significant decrease in the number of infected cells (FIG. 5F, albeit while also having a modest impact on parasite development, as measured by parasite area. See FIGS. 5G-H. Together, these data demonstrate that C4 reduces Plasmodium infection by interfering with parasite survival during development inside the host cell.

### Example 4. C4 Interacts with the UIS3-LC3 Complex

To determine whether the detrimental effect of C4 on Plasmodium survival was due to its impact on UIS3-LC3 interaction, Isothermal calorimetry (“ITC”) was performed using purified recombinant UIS3 and LC3 proteins. Direct binding of UIS3 and LC3 was confirmed with ITC. See FIG. 6A. With respect to adding C4 to the complex, ITC analysis showed a 10-fold higher affinity of C4 to the UIS3-LC3 complex (K_{d}=0.241±0.0011 μM), in comparison to the direct interaction of UIS3 to LC3 (K_{d}=2.15±0.121 μM). See FIGS. 6A-B. This change in K_{d} value indicated that C4 binds to the UIS3-LC3 complex, and can compete against the UIS3-LC3 interaction.

To perform these studies, His-mycUIS3 and GST-His LC3 were expressed in BL21(DE3) pLysS cells, cultured at 37° C. and the protein was induced with 0.3 mM IPTG at an OD600 of 0.8. After IPTG induction the cells were cultured at 18° C. for 16 hours. The cells were pelleted and resuspended in lysis buffer containing 50 mM Sodium Phosphate at pH 8, 300 mM NaCl, 10 mM Imidazole, 2 mM PhenylMethylSulphonyl Fluoride (PMSF) and Protease inhibitor cocktail (Roche). The proteins were purified by passing the lysate through His-trap Ni-charged columns (GE healthcare). The eluted proteins were proteolytically cleaved overnight with thrombin for myc-UIS3 and rTEV for LC3 at 4° C. to remove the tags (His tag from His-myc-UIS3 and GST-His tag from GST-His LC3). The proteins were then passed through a superdex-75 PG column (GE Healthcare). The proteins were finally concentrated to 10 mg/ml concentration in a final buffer containing 50 mM Sodium Phosphate and 150 mM Sodium Chloride at pH 6.5.

ITC experiments were performed using MicroCal-ITC 200 (GE healthcare) at 25° C. The data were analyzed using MicroCal ITC-origin analysis software based on one-site binding reaction. For direct interaction studies, a 10 μM concentration of pfUIS3 was titrated against 0.100 μM of LC3. For competition assay, a saturated solution of UIS3 with LC3 was titrated against 100 μM of C4. C4 was resuspended in the same final buffer (with 2% DMSO) in which UIS3 and LC3 was dissolved.

Nuclear magnetic resonance (NMR) studies were also conducted. For those experiments, the E. coli BL21 with the corresponding constructs were cultured in Luria broth overnight at 37° C. The cells were harvested and resuspended in 15N M9 media containing (3 g/l KH2PO4; 6 g/l Na2HPO4; 0.5 g/l NaCl; 1 g/l NH4Cl; 2 g/l D-Glucose; 1 g/l 15NH4Cl) and cultured at 37° C. till it reached an OD600 of 0.8, followed by Isopropyl β-D-1-thiogalactopyranoside (IPTG) induction. The proteins were then purified with the standard protocol as mentioned above.

The NMR samples containing 0.25 mM of either labelled LC3 or UIS3 was titrated against a titrant containing 1 mM of ligand in buffer containing 50 mM Sodium Phosphate, 150 mM NaCl, and pH 6.5 with 90% H2O:10% D2O. The experiments were performed on 800 or 600 MHz Bruker Avance III HD spectrometer with a cryoprobe at 298K. The backbone assignments for both the proteins were done using standard triple resonance spectroscopy experiments consisting of CBCA(CO)NH, HNCACB, HNCO, and HN(CA)CO. All the NMR data were processed using NMRpipe (Delaglio et al., 1995) and analyzed using SPARKY (Lee, Tonelli and Markley, 2015). After peak picking and data analysis the chemical shift perturbation (CSP) was plotted using the formula: [(δH Wt−δ H mutant)2+((δN Wt−δ N mutant)/25)2] ½ where δH and δN are the chemical shift of the amide hydrogen and nitrogen, respectively.

### Example 5. The Anti-Parasitic Activity of C4 Depends on Host Cell Autophagy

To test the hypothesis that high-affinity binding of C4 to the UIS3-LC3 complex could interfere with the function of the UIS3-LC3 complex to cause a decrease Plasmodium infection by interfering with the parasite's ability to evade host cell autophagy, genetic and chemicals tools were used to assess the anti-parasitic activity of C4 in autophagy-competent and autophagy-deficient cells. The results showed that C4 only reduced Plasmodium infection in cells with a functional autophagy pathway. Indeed, C4 did not cause any reduction in infection in Atg5^{-/-} mouse embryo fibroblast cells (“MEFs”), as they do not have a functional autophagy pathway. See FIG. 7A The impact on infection in C4-treated Atg5^{+/+} MEFs, which were used as controls, was comparable to that observed for infected Huh7 cells. See FIG. 7A Moreover, treatment of Huh7 cells with chloroquine, which is a known autophagy flux inhibitor, rescued the C4 effect, as determined by the number of infected hepatocytes (FIGS. 4b and c). See FIG. 7B-C These results demonstrated that C4 impacted infection via the host cell autophagy machinery.

MEFs used in these studies were obtained from the Riken BioResource Research Center (Atg5^{+/+} (RCB2710) and Atg5^{-/-} (RCB2711). (Kuma et al.) MEFs were cultured in DMEM supplemented as described above for Huh7 cells. MEFs were validated through quantitative reverse transcription PCR.

### Example 6. C4 does not Interfere with the Intrinsic Host Autophagy Pathway

As C4 acts on a protein-protein interaction at the host-parasite interface, the effect of C4 administration on host cell normal autophagy activity was determined. To that end, autophagy was induced in HeLa cells in the presence and absence of C4. The results showed that C4 does not cause any impairment in the host cells' ability to efficiently respond to an exogenous autophagy stimulus. See FIGS. 8A-B.

To perform the foregoing study, HeLa cells (ATCC), which had been maintained under standard culture conditions in DMEM medium supplemented with 10% FCS, 1% glutamine, 1% penicillin/streptomycin, were treated with 1 μM C4 or 0.0001% DMSO (control) for 24 h, and then changed from normal growth medium to EBBS to induce amino-acid starvation dependent autophagy. After 7 h, cells were collected in lysis buffer (50 mM NaCl, 50 mM Tris-Cl pH 8, 5 mM EDTA pH 8, 1% Triton X-100, protease inhibitor) and analysed by western blot using the following antibodies: p62 (rabbit polyclonal, Sigma-Aldrich, P0067, 1:1000) and gamma-tubulin (mouse monoclonal, Sigma-Aldrich, T5326, 1: 10,000). p62 levels were measured by quantifying the ratio of p62 to tubulin signals using Image Lab.

SEQUENCE LISTING
UIS3 P. falciparum
SEQ ID NO. 1
MKVSKLVLFAHIFFIINILCQYICLNASKVNKKGKIAEEKKRKN
IKNIDKAIEEHNKRKKLIYYSLIASGAIASVAAILGLGYYGYKK
SREDDLYYNKYLEYRNGEYNIKYQDGAIASTSEFYIEPEGINKI
NLNKPIIENKNNVDVSIKRYNNFVDIARLSIQKHFEHLSNDQKD
SHVNNMEYMQKFVQGLQENRNISLSKYQENKAVMDLKYHLQKVY
ANYLSQEEN
PfUIS3-HA forward primer
SEQ ID NO. 2
ATGGGCCCATGAAGGTCTCTAAATTAGTCTTG
PfUIS3-HA reverse primer
SEQ ID NO. 3
ATGCGGCCGCTTATGCATAATCTGGTACATCATATGGATATGCA
TAATCTGGTACATCATATGGATAGTTCTCTTCT
TGAGATAAATAATTAG
PbUIS3 5′ UTR forward primer
SEQ ID NO. 4
TAAAGCTTAAGGATTATATTTTATAATGTTTCAC
PbUIS3 5′ UTR reverse primer
SEQ ID NO. 5
ATGGGCCCTTTTATACACTTTCATATATTTGTTAT
PbUIS3 3′ UTR forward primer
SEQ ID NO. 6
TAGGTACCATGTTTGTGTAACATCATTTATAG
PbUIS3 3′ UTR reverse primer
SEQ ID NO. 7
ATAAGCTTTTCATATATCAATTTTCAAATTG
PbUIS4 3′ UTR forward primer
SEQ ID NO. 8
ATGCGGCCGCTTCATTATGAGTAGTGTAATTCAG
PbUIS4 3 UTR reverse primer
SEQ ID NO. 9
ATGAATTCGCATACAACATATGTAAAAAAG
PfUIS3-HA Recombinant locus 5′ integration
forward primer
SEQ ID NO. 10
ATTATATTGTGCTATAAAGCG
PfUIS3-HA Recombinant locus 5′ integration
reverse primer
SEQ ID NO. 11
GCATTTAAACAAATATATTGACATAAGAT
WT locus forward primer
SEQ ID NO. 12
TAAAGCTTAAGGATTATATTTTATAATGTTTCAC
WT locus reverse primer
SEQ ID NO. 13
GCAGCTAGTTTCACATTATCCATAAATAT
Control (PbUIS3 5′ UTR) forward primer
SEQ ID NO. 14
TAAAGCTTAAGGATTATATTTTATAATGTTTCAC
Control (PbUIS3 5′ UTR) reverse primer
SEQ ID NO. 15
ATGGGCCCTTTTATACACTTTCATATATTTGTTAT

### REFERENCES

1. Yu, L., Chen, Y. & Tooze, S. A. Autophagy pathway: Cellular and molecular mechanisms. Autophagy 14, 207-215 (2018).
2. Levine, B., Mizushima, N. & Virgin, H. W. Autophagy in immunity and inflammation. Nature 469, 323-335 (2011).
3. Wu, Y. W. & Li, F. Bacterial interaction with host autophagy. Virulence 10, 352-362 (2019).
4. Choi, Y., Bowman, J. W. & Jung, J. U. Autophagy during viral infection—a double-edged sword. Nature reviews. Microbiology 16, 341-354 (2018).
5. Latre de Late, P. et al. Apicomplexan autophagy and modulation of autophagy in parasite-infected host cells. Biomedical journal 40, 23-30 (2017).
6. Evans, R. J., et al. The Interplay of Host Autophagy and Eukaryotic Pathogens. Frontiers in cell and Dev Bio 6, 118 (2018).
7. Ashley, E. A., Pyae Phyo, A. & Woodrow, C. J. Malaria. Lancet 391, 1608-1621 (2018).
8. WHO World malaria report 2018. (World Health Organization, 2018).
9. Vaughan, A. M. & Kappe, S. H. I. Malaria Parasite Liver Infection and Exoerythrocytic Biology. Cold Spring Harbor perspectives in medicine 7 (2017).
10. Liehl, P. et al. Host-cell sensors for Plasmodium activate innate immunity against liver-stage infection. Nature medicine 20, 47-53 (2014).
11. Real, E. et al. Plasmodium UIS3 sequesters host LC3 to avoid elimination by autophagy in hepatocytes. Nature microbiology 3, 17-25 (2018).
12. Thieleke-Matos, C. et al. Host cell autophagy contributes to Plasmodium liver development. Cellular microbiology 18, 437-450 (2016).
13. Schmuckli-Maurer, J. et al. Inverted recruitment of autophagy proteins to the Plasmodium berghei parasitophorous vacuole membrane. PloS one 12, e0183797 (2017).
14. Lopes da Silva, M. et al. The host endocytic pathway is essential for Plasmodium berghei late liver stage development. Traffic 13, 1351-1363 (2012).
15. Agop-Nersesian, C. et al. Shedding of host autophagic proteins from the parasitophorous vacuolar membrane of Plasmodium berghei. Scientific reports 7, 2191 (2017).
16. Ogawa, M. et al. Escape of intracellular Shigella from autophagy. Science 307, 727-731 (2005).
17. Romano, P. S., Gutierrez, M. G., Beron, W., Rabinovitch, M. & Colombo, M. I. The autophagic pathway is actively modulated by phase II Coxiella burnetii to efficiently replicate in the host cell. Cellular microbiology 9, 891-909 (2007).
18. Orvedahl, A. et al. HSV-1 ICP34.5 confers neurovirulence by targeting the Beclin 1 autophagy protein. Cell host & microbe 1, 23-35 (2007).
19. Muniz-Feliciano, L. et al. Toxoplasma gondii-induced activation of EGFR prevents autophagy protein-mediated killing of the parasite. PLoS pathogens 9, e1003809 (2013).
20. Prado, M. et al. Long-term live imaging reveals cytosolic immune responses of host hepatocytes against Plasmodium infection and parasite escape mechanisms. Autophagy 11, 1561-1579 (2015).
21. Zhao, C. et al. The rodent malaria liver stage survives in the rapamycin-induced autophagosome of infected Hepa1-6 cells. Scientific reports 6, 38170 (2016).
22. Wacker, R. et al. LC3-association with the parasitophorous vacuole membrane of Plasmodium berghei liver stages follows a noncanonical autophagy pathway. Cellular microbiology 19 (2017).
23. Meis, J. F., Verhave, J. P., Jap, P. H., Sinden, R. E. & Meuwissen, J. H. Ultrastructural observations on the infection of rat liver by Plasmodium berghei sporozoites in vivo. The Journal of protozoology 30, 361-366 (1983).
24. Lingelbach, K. & Joiner, K. A. The parasitophorous vacuole membrane surrounding Plasmodium and Toxoplasma: an unusual compartment in infected cells. Journal of cell science 111 (Pt 11), 1467-1475 (1998).
25. Nyboer, B., Heiss, K., Mueller, A. K. & Ingmundson, A. The Plasmodium liver-stage parasitophorous vacuole: A front-line of communication between parasite and host. International journal of medical microbiology: IJMM 308, 107-117 (2018).
26. Mueller, A. K., Labaied, M., Kappe, S. H. & Matuschewski, K. Genetically modified Plasmodium parasites as a protective experimental malaria vaccine. Nature 433, 164-167 (2005).
27. Farre, J. C. & Subramani, S. Mechanistic insights into selective autophagy pathways: lessons from yeast. Nature reviews. Molecular cell biology 17, 537-552 (2016).
28. Ganley, I. G., Wong, P. M., Gammoh, N. & Jiang, X. Distinct autophagosomal-lysosomal fusion mechanism revealed by thapsigargin-induced autophagy arrest. Molecular cell 42, 731-743 (2011).
29. Morel, E. et al. Autophagy: A Druggable Process. Annual review of pharmacology and toxicology 57, 375-398 (2017).
30. Derbyshire, E. R., Mota, M. M. & Clardy, J. The next opportunity in anti-malaria drug discovery: the liver stage. PLoS pathogens 7, e1002178 (2011).
31. Boonhok, R. et al. LAP-like process as an immune mechanism downstream of IFN-gamma in control of the human malaria Plasmodium vivax liver stage. Proceedings of the National Academy of Sciences of the United States of America 113, E3519-3528 (2016).
32. Cappellini, M. D. & Fiorelli, G. Glucose-6-phosphate dehydrogenase deficiency. Lancet 371, 64-74 (2008).
33. Watson, J. et al. Implications of current therapeutic restrictions for primaquine and tafenoquine in the radical cure of Vivax malaria. PLoS neglected tropical diseases 12, e0006440 (2018).
34. Kuma, A. et al. The role of autophagy during the early neonatal starvation period. Nature 432, 1032-1036 (2004).
35. Janse, C. J., Ramesar, J. & Waters, A. P. High-efficiency transfection, and drug selection of genetically transformed blood stages of the rodent malaria parasite Plasmodium berghei. Nature protocols 1, 346-356 (2006).
36. Li, H., Leung, K. S., Ballester, P. J. & Wong, M. H. istar: a web platform for large-scale protein-ligand docking. PloS one 9, e85678 (2014).
37. Pronk, S. et al. GROMACS 4.5: a high-throughput and highly parallel open source molecular simulation toolkit. Bioinformatics 29, 845-854 (2013).
38. Ploemen, I. H. et al. Visualisation and quantitative analysis of the rodent malaria liver stage by real time imaging. PloS one 4, e7881 (2009).
39. Hanson, K. K. et al. Torins are potent antimalarials that block replenishment of Plasmodium liver stage parasitophorous vacuole membrane proteins. Proceedings of the National Academy of Sciences of the United States of America 110, E2838-2847 (2013).
40. Delaglio, F. et al. (1995) ‘NMRPipe: A multidimensional spectral processing system based on UNIX pipes’, Journal of Biomolecular NMR, 6(3), pp. 277-293. doi: 10.1007/BF00197809.
41. Lee, W., Tonelli, M. and Markley, J. L. (2015) ‘NMRFAM-SPARKY: Enhanced software for biomolecular NMR spectroscopy’, Bioinformatics, 31(8), pp. 1325-1327. doi: 10.1093/bioinformatics/btu830.

What is claimed:

1. A method for treating a Plasmodium parasite infection in subject in need thereof, comprising administering a therapeutically effective dose of an inhibitor of the direct interaction between host cell Microtubule-associated Protein 1A/1B-light chain 3 (LC3) and parasite Upregulated In Infective Sporozoites 3 (UIS3).

2. The method of claim 1, wherein the inhibitor inhibits sequestration of LC3 on PVMs by UIS3.

3. The method of claim 1, wherein inhibition of the direct interaction between host cell LC3 and parasite UIS3 by the inhibitor facilitates elimination of the parasite from the host cell by an autophagy-dependent process.

4. The method of claim 1, wherein the Plasmodium parasite is P. falciparum, P. vivax, P. ovale, P. malariae, or P. knowlesi.

5. The method of claim 1, wherein the inhibitor binds UIS3.

6. The method of claim 5, wherein binding of the inhibitor to UIS3 blocks direct interaction between UIS3 and LC3.

7. The method of claim 6, wherein the Plasmodium parasite is P. falciparum, and binding of the inhibitor to UIS3 blocks the interaction of UIS3 with LC3 at one or more of the following UIS3 amino acid positions: N181, E183, M182, K213, and Q217.

8. The method of claim 1, wherein the inhibitor is a member of the phenyloxadiazole class of small molecule compounds.

9. The method of claim 8, wherein the inhibitor comprises an oxadiazole ring connected to a tri-fluoro-methyl-benzene and an N-alkyl-piperazine, linked to a nitrile derivative of benzoic acid.

10. The method of claim 9, wherein the inhibitor is (4-{[4-(4-{5-[3-(trifluoromethyl) phenyl]-1,2,4-oxadiazol-3-yl}benzyl)piperazino]carbonyl}benzonitrile (“C4”).

11. The method of claim 1, wherein the therapeutically effective dose of the inhibitor is administered orally, parenterally, subcutaneously, or transdermally.

12. The method of claim 11, wherein the therapeutically effective dose of the inhibitor is administered transdermally using a dermal-type patch or article.

13. The method of claim 1, wherein the therapeutically effective dose of the inhibitor is administered to the subject in combination with at least one other antimalarial drug.

14. The method of claim 1, wherein the at least one other antimalarial drug is: an AMPK activation agent, Quinine or a quinine-related agents; Chloroquine; Amodiaquine; Pyrimethamine; Atovaquone; Artemisinin or a artemisinin derivative; Halofantrine; Doxycycline; Clindamycin; 8-aminoquinoline or an 8-aminoquinoline derivative drug; a Dipeptidyl peptidase-4 (“DPP-4”) inhibitor; or any combination thereof.

15. The method of claim 14, wherein: the AMPK activation agent is a biguanide; the 8-aminoquinoline derivative drug is bulaquine, pamaquine, primaquine, or tafenoquine; and the DPP-4 inhibitor is sitagliptin, vildagliptin, saxagliptin, linagliptin, gemigliptin, anagliptin, teneligliptin, alogliptin, trelagliptin, omarigliptin, evogliptin, gosogliptin, dutogliptin, or berberine.

16. The method of claim 15, wherein the biguanide is metformin.


SEQUENCE LISTING (15 sequences)

SEQ ID NO: 1 - protein, 229 aa, Plasmodium falciparum
MKVSKLVLFAHIFFIINILCQYICLNASKVNKKGKIAEEKKRKNIKNIDKAIEEHNKRKKLIYYSLIASGAIASVAAILGLGYYGYKKSREDDLYYNKYLEYRNGEYNIKYQDGAIASTSEFYIEPEGINKINLNKPIIENKNNVDVSIKRYNNFVDIARLSIQKHFEHLSNDQKDSHVNNMEYMQKFVQGLQENRNISLSKYQENKAVMDLKYHLQKVYANYLSQEEN

SEQ ID NO: 2 - DNA, 32 bp, Artificial Sequence
  HA tagged primer
atgggcccatgaaggtctctaaattagtcttg

SEQ ID NO: 3 - DNA, 93 bp, Artificial Sequence
  HA tagged primer
atgcggccgcttatgcataatctggtacatcatatggatatgcataatctggtacatcatatggatagttctcttcttgagataaataattag

SEQ ID NO: 4 - DNA, 34 bp, Plasmodium berghei
taaagcttaaggattatattttataatgtttcac

SEQ ID NO: 5 - DNA, 35 bp, Plasmodium berghei
atgggcccttttatacactttcatatatttgttat

SEQ ID NO: 6 - DNA, 32 bp, Plasmodium berghei
taggtaccatgtttgtgtaacatcatttatag

SEQ ID NO: 7 - DNA, 31 bp, Plasmodium berghei
ataagcttttcatatatcaattttcaaattg

SEQ ID NO: 8 - DNA, 34 bp, Plasmodium berghei
atgcggccgcttcattatgagtagtgtaattcag

SEQ ID NO: 9 - DNA, 30 bp, Plasmodium berghei
atgaattcgcatacaacatatgtaaaaaag

SEQ ID NO: 10 - DNA, 21 bp, Artificial Sequence
  HA tagged primer
attatattgtgctataaagcg

SEQ ID NO: 11 - DNA, 29 bp, Artificial Sequence
  HA tagged primer
gcatttaaacaaatatattgacataagat

SEQ ID NO: 12 - DNA, 34 bp, Plasmodium berghei; identical to SEQ ID NO: 4

SEQ ID NO: 13 - DNA, 29 bp, Plasmodium berghei
gcagctagtttcacattatccataaatat

SEQ ID NO: 14 - DNA, 34 bp, Plasmodium berghei; identical to SEQ ID NO: 4

SEQ ID NO: 15 - DNA, 35 bp, Plasmodium berghei; identical to SEQ ID NO: 5